(19) United States
(12) Patent Application Publication
(10) Pub. No.: US 2011/0238028 A1
(43) Pub. Date: Sep. 29, 2011

(54) TAMPON WITH MODIFIED CONSTRICTED WITHDRAWAL END

(75) Inventor: Steven Smet, Zele (BE)

(73) Assignee: ONTEX HYGIENEARTIKEL DEUTSCHLAND GMBH

(21) Appl. No.: 13/130,971

(22) PCT Filed: Dec. 14, 2009
(86) PCT No.: PCT/EP09/67047

§ 371 Date: May 24, 2011

(30) Foreign Application Priority Data
Dec. 16, 2008 (EP) 08171801.7

(51) Int. Cl. A61F 13/20; B29C 43/02

(52) U.S. Cl. 604/385.17; 264/239

(57) ABSTRACT

A tampon is described which includes a generally elongated body. The elongated body includes an insertion end, a withdrawal end and a central portion extending therebetween. The withdrawal end is constricted. The constricted withdrawal end includes one or more radially pressed grooves.

### FIELD OF THE INVENTION

The present invention relates to tampons, particularly tampons for insertion into a mammalian body cavity, such as catamenial tampons. More in particular, the invention concerns improvements to the withdrawal end of such tampons.

### BACKGROUND OF THE INVENTION

A tampon comprising a constricted withdrawal end and advantages thereof are known from EP 1 481 656 A1. The constricted withdrawal end reduces the contact of the tampon surface with the surrounding tissues when inserting the tampon into a body cavity, thus causing less irritation and discomfort. Also, owing to its constriction, the withdrawal end of the tampon may expand relatively less than the remaining portions of the tampon's body when accepting fluids, thereby facilitating withdrawal of the tampon after use.

Whereas EP 1 481 656 A1 discloses a tampon comprising longitudinal grooves extending within the central portion of the tampon's body, the withdrawal end of said tampon is not provided with such grooves nor with other radially pressed areas.

The present Applicant has found that notwithstanding the known advantages of tampons with a constricted withdrawal end as disclosed in EP 1 481 656 A1, there remains room for further improvement of such tampons.

For example, the outer cover commonly provided on the absorbent core of tampons may show a propensity to detach from said absorbent core within the constricted withdrawal end of the tampon. This may increase the risk of fibre loss from the absorbent core, as well as cause increased leakage and less comfortable removal of the tampon from the body cavity following use.

Furthermore, due to the frequently non-random fibre orientation inherent in the production process for the absorbent material forming the absorbent core of tampons, the withdrawal end of tampons may often represent a relatively weaker section more prone to fibre loss. Such fibre loss may be more pronounced at the constricted withdrawal end of tampons, where the volume of displaced material tends to push the material away from the tampon core towards the withdrawal end. Fibre loss can cause discomfort, generally reduces hygiene and may increase the risk of infections or other adverse reactions.

In addition, memory effects occurring due to the inherent elasticity of fibres of the compressed absorbent material of the tampon core can cause the tampon to at least partially revert or ‘bounce back’ to the shape and volume it had as a tampon blank before compression. Such memory effects become particularly important when the tampon is exposed to moisture. Said memory effects may understandably reduce the advantages of the constricted withdrawal end.

Moreover, consumers may experience it as more demanding to securely grasp and hold such tampons at their constricted withdrawal end. Hence, on occasion such tampons tend to sway away from or lose proper contact with a consumer's fingertip during insertion into a body cavity.

Thus, prior art tampons with a constricted withdrawal end have not been fully satisfactory and their performance can benefit from further improvements.

### SUMMARY OF THE INVENTION

Having conducted assessments, the present Applicant has realised that several characteristics of tampons comprising a constricted withdrawal end can be markedly improved when the constricted withdrawal end of said tampons is provided with one or more radially pressed grooves.

For example, the provision of said grooves can improve the attachment of the outer cover at the withdrawal end of the tampon. As known per se, tampons are commonly provided with an outer cover to reduce fibre loss and to provide a smooth and soft surface increasing the comfort during the use of the tampon. The properties of the material of the outer cover are usually fairly distinct from the properties of the material of the absorbent core of the tampon. For example, a tampon core (such as, e.g., commonly viscose-based) reacts differently under a compressing or densifying process than a tampon outer cover (such as, e.g., commonly polyethylene/polypropylene based)—for example, the former may have less memory effect than the latter. As a result, the outer cover can detach from the core more easily in the constricted withdrawal end of the tampon, especially when this takes up fluid or moisture (FIG. 4A), leading to disassociation of (hydrogen and other) bonds between the materials. The Applicant has realised that by providing radially pressed grooves in the constricted withdrawal end of the tampon, a mechanical entanglement or association between the outer cover and the absorbent core is created or improved, ensuring a tighter fit of the outer cover to the absorbent core even in wet conditions (FIG. 4B).

Furthermore, conventional tampons having a fibre-based absorption core are commonly manufactured according to the following steps: individual absorbing fibres are processed into a coherent material; a strip of this material is cut, wound up to form a tampon blank, and then compressed to form the final tampon. A frequently employed method to transform individual fibres into a coherent material may involve carding as known per se. However, the fibre orientation in the resulting material will usually not be completely random: instead, owing to the carding (or analogous) process, more fibres may have an orientation in the machine direction (MD) than in cross direction (CD), and consequently the CD strength may be less than the MD strength (FIG. 5A). A great proportion of tampons (e.g., substantially all digital tampons and many applicator tampons) are manufactured by winding the strip of material along a CD axis, resulting in a tampon blank with—due to the above described relative CD weakness—outer ends that are comparably weak and prone to material loss, e.g., fibre loss (FIG. 5B). The subsequent radial compression of the tampon blank does not completely eliminate this problem. Especially the withdrawal end of the tampon remains a comparably weak section subject to fibre loss. As described, this can be more pronounced in tampons with constricted withdrawal ends. The Applicant has realised that by providing radially pressed grooves in the constricted withdrawal end of the tampon, the fibre orientation is altered, and even fibre deformation can be achieved, whereby fibre entanglement is generally significantly improved (FIG. 5C). Also the local fibre density profile is changed. These combined effects provide for a stronger tampon withdrawal end, having less CD weakness and more resistant to fibre loss or material detachment.

In addition, the Applicant has realised that provision of radially pressed grooves within the constricted withdrawal end of the tampon can significantly reduce the memory effect of the material, (e.g., by providing void space allowing expansion in the groove structure and/or providing improved entanglement of the fibres), and thereby more adequately preserve the constricted shape of said withdrawal end and the advantages associated therewith.

The present Applicant has also realised that the ease of manipulation of tampons comprising a constricted withdrawal end can be markedly improved when the constricted withdrawal end of said tampons is provided with one or more radially pressed grooves.

Without limitation, the Applicant considers that the latter advantages of the inclusion of the one or more radially pressed grooves within the restricted withdrawal end of tampons may occur by virtue of one or more of the following effects.

In particular, the radially pressed grooves delineate adjacent, radially outwardly extending ribs. Said grooves and ribs thus represent areas of respectively more and less compression or density of the absorbent material of the tampon. Due to this non-uniform compression, the ribs—which constitute the portions mainly contacted by a consumer's fingers—are relatively impressionable and can advantageously conform to the pressure of, and thereby engage more closely and extensively with, the consumer's fingers. This contrasts with the uniformly compressed withdrawal end of prior art tampons, which was comparably rigid and tended to slide away between the consumer's fingers. This advantage can also occur when the present tampons are used in conjunction with a tampon applicator, whereby the respective axial edge of the of inner (ejector) tube of such applicator can more securely engage with the withdrawal end of the tampon, thereby preventing the re-entry of the tampon into the inner tube upon ejection. The above effects can be even more pronounced where the one or more grooves or portion(s) of said one or more grooves in the withdrawal end extend at an angle with the longitudinal axis of the tampon. Such orientation of the grooves reduces the uniformity of the surface of the withdrawal end, whereby a better contact or frictional engagement can be obtained with a consumer's fingers or with the ejector tube of a tampon applicator.

Moreover, and particularly when the constricted withdrawal end of the tampon comprises a recess configured to receive a consumer's fingertip, the radially pressed grooves can allow some degree of radially outward extension or opening out of the constricted withdrawal end in response to the force applied by the consumer's finger, whereby the constricted withdrawal end can closer conform to and more securely accommodate diverse sizes of consumer's fingertips.

In addition, particularly when the grooves of the withdrawal end are formed by radial pressure greater than any grooves optionally provided on the remainder of the tampon body, in particular on its central section, the withdrawal end can display a relatively lower expansion capacity compared to remainder of the tampon body, in particular to its central section. Hereby, the withdrawal end can have an advantageously smaller size after the tampon has absorbed liquids and can facilitate the withdrawal of the tampon after use.

In view of the above advantages realised by the Applicant, an aspect of the invention relates to a tampon having a generally elongated body comprising an insertion end, a withdrawal end and a central portion extending therebetween, wherein said withdrawal end is constricted, characterised in that said constricted withdrawal end comprises one or more radially pressed grooves.

Further aspects of the invention relate to a method for manufacturing said tampon; an apparatus specifically designed for manufacturing said tampon; the use of said tampon for absorbing a liquid such as discharge; and an assembly comprising a tampon applicator and a tampon as taught herein.

### BRIEF DESCRIPTION OF FIGURES

The invention will be described in the following in greater detail by way of example only and with reference to the attached drawings of non-limiting embodiments of the invention, in which:

FIG. 1 illustrates a side view of the tampon according to an embodiment of the invention.

FIG. 2 illustrates a side view of the tampon according to various embodiments of the invention.

FIG. 3 illustrates a detailed view of exemplary shapes of the withdrawal end of the tampons according to various embodiments the invention.

FIG. 4A illustrates a side-view of a cross-section along the longitudinal axis of a tampon not comprising radially pressed grooves within its withdrawal end 3. The occurrence of the undesirable detachment of the outer cover 15 from the absorbent core 14 of the tampon in the area of the withdrawal end 3 of such tampon is schematically shown (arrow).

FIG. 4B illustrates a partial view of a transverse cross section through the withdrawal end 3 of a tampon according to an embodiment of the invention. The improved mechanical entanglement or association between the outer cover 15 and the absorbent core 14 resulting from the provision of the grooves 10 can be appreciated.

FIG. 5A schematically illustrates a top-view of a strip of a web of tangled fibrous material used to produce a tampon blank. The feature 16 schematically denotes the fact that the individual fibres in said strip may be preferentially oriented in the machine direction (MD), thereby causing comparable weakness in the cross-direction (CD).

FIG. 5B shows a tampon blank obtained by rolling or folding the strip of fibrous material illustrated in FIG. 5A on itself. The proximal 5 and distal 6 directions of the tampon blank are shown. The preferential orientation of fibres is depicted by feature 16.

FIG. 5C illustrates a partial view of a transverse cross section through the withdrawal end 3 of a tampon according to an embodiment of the invention. Feature 16 schematically captures the altered fibre orientation or even fibre deformation, and improved entanglement of the fibres, resulting in greater strength in the areas (arrowheads) of the grooves 10, as compared to the remaining areas.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, term definitions may be included to better appreciate the teaching of the present invention.

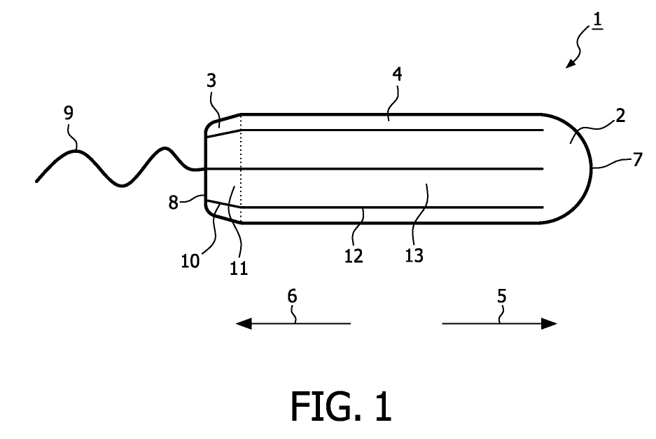

In FIG. 1, the reference sign 1 generally denotes a tampon having a generally elongated body comprising a insertion end 2, a withdrawal end 3 and a central portion 4 extending therebetween, wherein said withdrawal end 3 is constricted. Arrows 5 and 6 denote axial directions referred to herein as proximal and distal, respectively. Accordingly, the insertion end 2 is denoted herein as the proximal-most part of the tampon, whereas the withdrawal end 3 is denoted herein as the distal-most part of the tampon.

The term “tampon” broadly encompasses any type of absorbent structure used for inserting into openings, orifices, cavities or canals with the purpose of absorbing liquid or moisture potentially present therein or discharged there from. For example, tampons may be configured and intended for inserting into a body cavity or canal of an animal, preferably mammal, more preferably human, e.g., to absorb secretions, fluid or moisture there from. For example, tampons may be inserted into the vaginal canal, urinal canal, rectum, ear canal, nasal canal or throat. Alternatively, tampons can also be used in such body orifices to administer substances such as drugs, microflora or moisture, to assist in health improvement processes. In another example, tampons may be configured and intended for inserting within industrial installations, equipments or appliances, such as for instance tubing or piping.

A preferred tampon type are catamenial tampons, configured for insertion into the vaginal canal or cavity of mammal females, preferably human females, for absorbing and preventing the leakage of menstrual fluid.

Preferably, the tampons as intended herein may be self-sustaining shape tampons. The term “self-sustaining shape” tampon encompasses tampons which tend to retain their general shape and size before use. Typically, such tampons can be obtained when a tampon blank comprising absorbent material, e.g., is pressed or compressed and/or shaped such that it assumes and maintains said general shape and size. Self-sustaining shape tampons tend to have, before use, a high modulus of compression, low resiliency, high stability and buckling strength, and tend to be relatively small in cross-section in order to attain insertion, wearing and removal comfort. Self-sustaining shape tampons are presently in widespread use, and non-limiting, exemplary disclosures thereof include, e.g., GB 394,571, GB 490,024, DE 3,934,153 C2 and U.S. Pat. No. 5,911,712, incorporated by reference herein.

The shape and size of catamenial tampons as intended herein is chosen to be vaginally insertable. Typically, such tampons may have length of between about 20 mm and about 70 mm, preferably between about 35 mm and about 60 mm, more preferably up to about 55 mm, the length measured from and including the insertion end (top) of the tampon to the withdrawal end (base) of the tampon along its longitudinal axis, and not including in the measurement any over-wrap, secondary absorbent member, or withdrawal cord which may extend beyond the tampon's main absorbent material. Usually, the width of the tampons may be between about 5 mm and about 25 mm, preferably between about 8 mm and about 15 mm, more preferably between about 10 mm and about 14 mm, corresponding to the largest cross-sectional diameter (“cross-section” refers to a slice taken at right angles to the longitudinal axis of a tampon). The width may vary along the length of the tampon. Typically, the tampons may have a generally elongated shape, preferably a generally cylindrical shape. It will be understood by one of skill in the art that the shape need not, and preferably does not persist during actual use of the tampon. That is, once the tampon is inserted and begins to acquire fluid, the tampon may begin to expand and may lose its form.

The present disclosure pertains inter alia to digital tampons. The term “digital tampon” refers to a tampon which is intended to be inserted into the body cavity, especially vaginal canal, with the user's finger and without the aid of an applicator. The present disclosure can also be applied to tampons configured to be placed with the aid of an applicator. The applicator may be any known in the art, such as, e.g., a telescoping-tube type applicator, including the tube-and-plunger type or the compact type arrangements. The applicator may be plastic, paper, or any other suitable material, e.g., may be cardboard or moulded polyethylene.

Preferably, the tampons as intended herein may have a generally cylindrical shape. The term “generally cylindrical” does not refer necessarily to a right circular cylinder, but rather to a shape that can be generally approximated by a geometrical cylinder or a derivative thereof. Hence, the term embraces, inter alia, a typical cylinder form, a truncated cone form (a frustoconical shape), a barrel form (pineapple shape), oblate or partially flattened cylinder forms, curved cylinder forms, cylindrical forms with varying cross-sectional areas (e.g., hourglass shape, rivet shape, mushroom shape, bullet shape, etc.), and the like. It shall be appreciated that while a tampon may have an overall generally cylindrical shape, its surface may include profiles, such as, e.g., variably shaped grooves or ribs.

In one preferred connotation, the term “generally cylindrical” refers to the usual shapes of tampons, especially catamenial tampons, as well-known in the art.

The axially proximal insertion end 2 of the tampon 1 leads the insertion of the tampon 1 into a (body) cavity, whereas the axially distal withdrawal end 3 of the tampon 1 guides the withdrawal of the tampon 1 from the (body) cavity.

Preferably, the insertion end 2 of the tampon 1 may be constricted. The term “constricted” as used herein is generally synonymous to terms such as narrowed, tapered or pointed. Hence, as intended herein, the insertion end 2 is “constricted” when the insertion end 2 or a portion thereof comprises a plurality of cross-sectional (i.e., transverse cross-section) perimeters or cross-sectional diameters decreasing, e.g., gradually decreasing, in the direction towards the proximal extreme 7 of the insertion end 2. In an embodiment, the cross-sectional perimeters or cross-sectional diameters of the insertion end 2 are generally smaller than the cross-sectional perimeters or cross-sectional diameters of the central section 4 of the tampon body. In exemplary embodiments, the insertion end 2 may assume a round-dome shape, a mushroom-head shape, etc. Constriction of the insertion end 2 achieves easier insertion of the tampon into a body cavity.

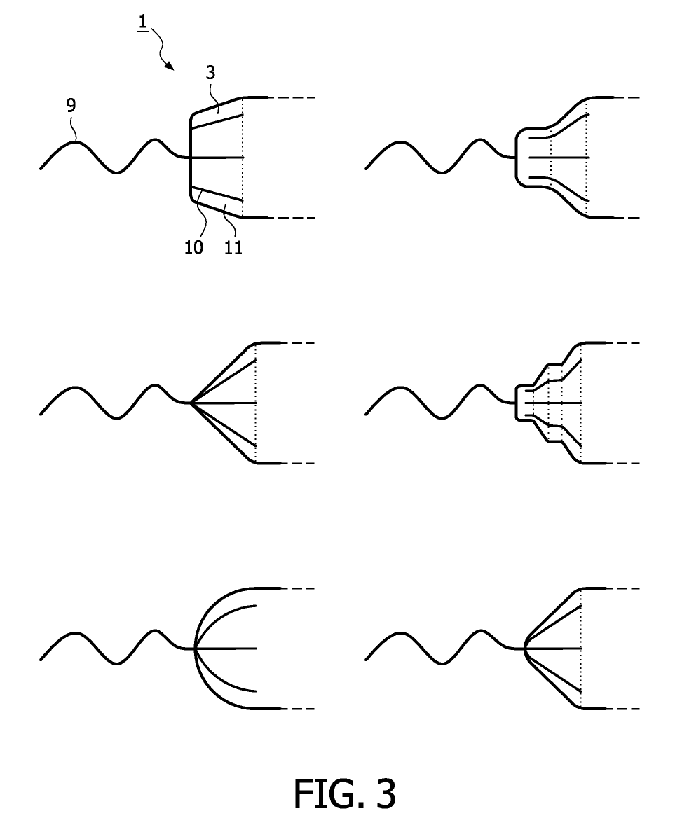

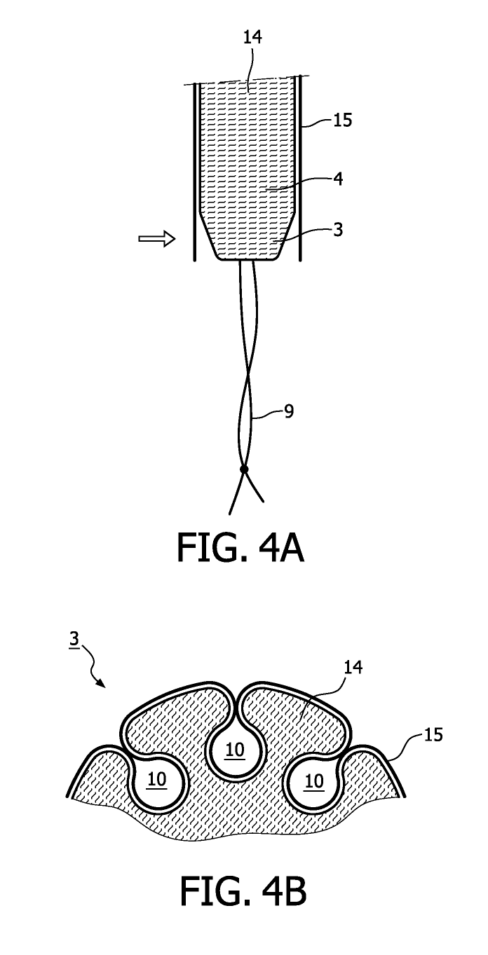

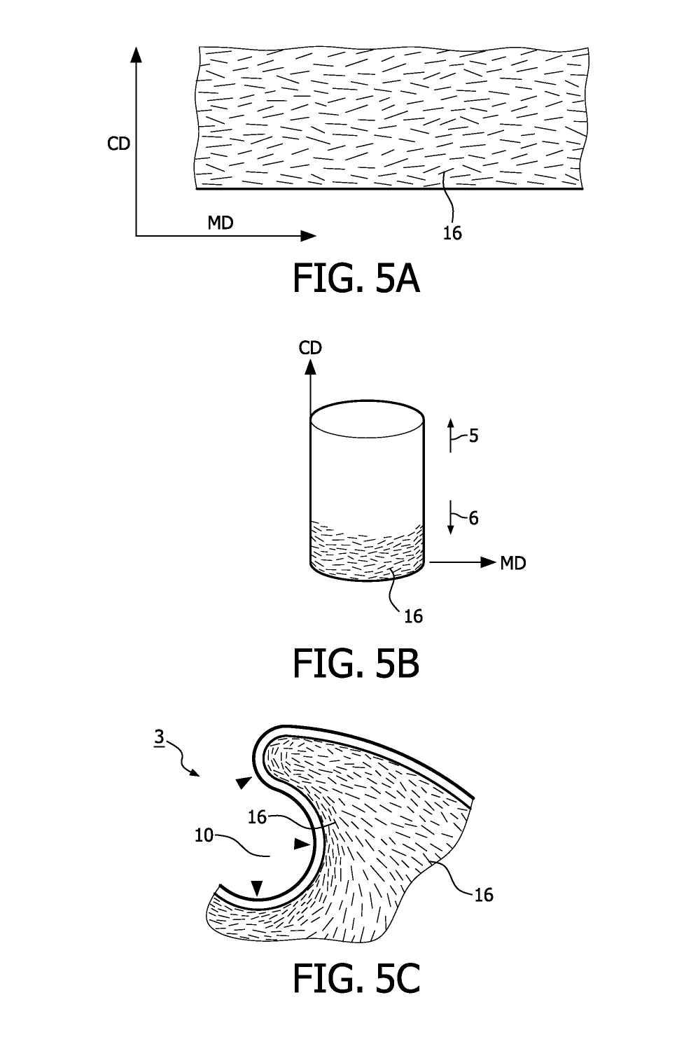

The withdrawal end 3 of the tampon 1 is constricted as denoted above. Hence, as intended herein, the withdrawal end 3 is “constricted” when the withdrawal end 3 or a portion thereof comprises a plurality of cross-sectional (i.e., transverse cross-section) perimeters or cross-sectional diameters decreasing, e.g., gradually decreasing, in the direction towards the distal extreme 8 of the withdrawal end 3. In an embodiment, the cross-sectional perimeters or cross-sectional diameters of the withdrawal end 3 are generally smaller than the cross-sectional perimeters or cross-sectional diameters of the central section 4 of the tampon body. In exemplary embodiments, the withdrawal end 3 may assume a conical shape, a frusto-conical shape, a round-dome shape, etc. Exemplary, non-limiting shapes of the withdrawal end 3 are illustrated in FIG. 3.

The cross-sectional diameter of the constricted withdrawal end 3 preferably may be generally smaller than 25 mm; preferably smaller than 15 mm, or smaller than 10 mm, or smaller than 5 mm. The cross-sectional diameters of the constricted withdrawal end 3 may preferably comprise between 0.1 and 0.95 times the cross-sectional diameter of the central portion 4 of the tampon body, e.g., between 0.3 and 0.8 times said cross-sectional diameter, or between 0.5 and 0.7 times said cross-sectional diameter. By means of example and not limitation, the cross-sectional diameter of the distal edge 8 of the withdrawal end 3 may be between 0.3 and 0.8 times the cross-sectional diameter of the central portion 4, and the cross-sectional diameters of more proximal portions of the withdrawal end 3 may lie between said values.

By means of example and not limitation, the insertion end 2 may represent the proximal-most portion of the tampon body having axial length of between about 1% and about 30% of the complete axial length of the tampon body, such as, e.g., between about 2% and about 25%, or between about 5% and about 15%, or between about 7% and about 10% of the complete axial length of the tampon body. In examples, the insertion end 2 may have an axial length of between about 5 mm and about 25 mm.

By means of example and not limitation, the constricted withdrawal end 3 may represent the distal-most portion of the tampon body having axial length of between about 1% and about 30% of the complete axial length of the tampon body, such as, e.g., between about 2% and about 25%, or between about 5% and about 15%, or between about 7% and about 10% of the complete axial length of the tampon body. In examples, the withdrawal end 3 may have an axial length of between about 5 mm and about 25 mm.

In embodiments, the insertion end 2 and the withdrawal end 3 may have approximately same axial lengths.

Further features of the tampon as taught herein can be generally envisaged by a skilled person.

For example, the tampon body generally comprises, consists essentially of or consists of absorbent, preferably hydrophilic material(s), representative, non-limiting examples of which include cellulosic materials, such as rayon, cotton, wood pulp, comminute wood pulp, creped cellulose wadding, tissue wraps and laminates, peat moss, and chemically stiffened, modified, or cross-linked cellulosic fibres; polymeric materials, such as polyester fibres, polyolefin fibres, absorbent foams, e.g., resilient polyurethane foam, absorbent sponges, super-absorbent polymers, absorbent gelling materials; formed fibres, such as capillary channel fibres and multi-limbed fibres; combination materials, such as, e.g., synthetic fibres and wood pulp, including co-formed fibrous structures (e.g., those materials described in U.S. Pat. No. 4,100,324); or any equivalent materials, or combinations or mixtures of above materials. As a non-limiting example, suitable fibre material includes Galaxy regenerate cellulosic fibres which are commercially available from Acordis Kehlheim Fibers, Germany.

The tampon body may comprise a liquid-pervious outer cover at least partly ensheathing the outer surface of the absorbent structure of the tampon. Such outer covers can reduce fibre loss from the absorbent structure of the tampon, and can also provide a smoother and pleasanter sensation upon insertion and withdrawal of the tampon. The term “liquid-pervious” denotes that the outer cover is capable of allowing liquids, fluids or moisture, either hydrophilic or hydrophobic, preferably at least hydrophilic, to penetrate readily through its thickness in at least one direction. Suitable outer covers include without limitation apertured or aperture-formed plastic films or non-woven webs. Preferably, the outer cover may substantially ensheathe at least the surface of the central section 4 of the tampon body, but it may also at least partly ensheathe the insertion 2 and/or withdrawal 3 ends of the tampon. Preferably, the insertion end 2 of the tampon is not covered by the outer cover, to ensure direct and rapid absorption at said insertion end 2.

Further, the tampon may comprise a withdrawal string 9 attached to its withdrawal end 3 to facilitate withdrawal of the tampon after use. The withdrawal string 9 is preferably flexible, hydrophobic, long enough to protrude from the body cavity when the tampon is in place, and of sufficient tensile strength to resist breaking during removal of the tampon. A withdrawal string may be, e.g., a single cord, a tape, or a plurality of strings. Exemplary, non-limiting materials which have worked well as withdrawal strings are cotton, rayon, hydrophobic cotton, hydrophobic polyester or a mixture hereof.

When the tampon is intended to be digitally inserted, it may be provided with a finger indent or recess at the withdrawal end 3 of the tampon to aid its insertion. A finger recess can be made, e.g., using a compression rod. Exemplary tampon finger indents are found, e.g., in U.S. Pat. No. 6,283,952, herein incorporated by reference.

The tampon may optionally include further features, especially features commonly employed with tampons in the art, such as, e.g., one or more surface markings, e.g., printed and/or embossed markings; one or more colours, e.g., applied by printing or impregnation; one or more chemical indicators capable of indicative colour change, e.g., for detecting and indicating medical condition of the user (such exemplary features are disclosed, e.g., in , WO 2006/099944 A1, p. 21, I. 6 to 30, herein incorporated by reference).

Typically, tampons as above may be made by folding or rolling or otherwise manipulating a strip of absorbent material, such as, e.g., a loosely associated fibrous web, into an elongated shape, e.g., a generally cylindrical shape, known as a “tampon blank” or “softwind” and compressing the softwind into the tampon's ultimate shape. As used herein, “compression” refers to the process of pressing, squeezing, compacting or otherwise manipulating the size, shape, and/or volume of a material to obtain a tampon having an insertable and preferably self-sustaining shape.

Usually, said compression or shaping may comprise at least a radial component, i.e., a component of circumferential compression directed towards the central longitudinal axis of the tampon. By means of example, the compression or shaping may be in any or all of the width direction, the radial direction, and the axial direction. Conditions and manners of compression of softwinds to obtain digital tampons (which usually comprise a densely compressed central core and relatively less compressed outer segments or ribs protruding from said central core) or tampons mainly intended for use with an applicator (which may usually be relatively uniformly compressed on cross-section) are well-known in the art.

The constriction of the withdrawal end 3 may be formed during the same compressing action that forms the overall body of the tampon and/or may be formed or further formed by one or more separate compressing actions. Similarly, the optional and preferred constriction of the insertion end 2 may be formed during the same compressing action that forms the overall body of the tampon and/or may be formed or further formed by one or more separate compressing actions. A further compressing action prior to, concurrently with or following the compressing action(s) that forms the constricted withdrawal end 3 of the tampon can create the optional finger recess in said withdrawal end 3.

The withdrawal string can be attached either to the softwind before compression or following compression to the resulting tampon. The outer cover may be preferably provided on the softwind before compression, such that it is compressed simultaneously with the absorbent material to the final tampon shape. Alternatively, an outer cover may be secured to the absorbent core following compression.

The constricted withdrawal end 3 of the present tampon comprises one or more radially pressed grooves 10. Such radially pressed grooves may be generally obtainable by suitable pressing the tampon or a precursor thereof (e.g., a tampon blank), wherein said pressing involves at least a radial component. In a transverse cross-section through the withdrawal end 3, the groove 10 defines a smaller radial distance of the outer surface of the tampon from its central longitudinal axis than is the radial distance of the outer surfaces adjacent to said groove 10. Hence, a groove 10 is generally flanked by ribs 11 radially relatively more protruding than the groove 10. For example, a groove 10 may define two adjacent ribs 11 radially relatively more protruding than said groove 10. Similarly, two nearby grooves 10, e.g., grooves parallel to one another, may define a radially relatively more protruding rib 11 in between. The ribs 11 thus represent areas of the tampon relatively less compressed than said grooves 10.

Preferably, the grooves 10 may be longitudinal, i.e., extending along at least a portion of the axial length of the withdrawal end 3 of the tampon 1. In the broadest meaning, a longitudinal groove may be any which, in the direction of its length, is not perpendicular to the longitudinal axis of the tampon.

Preferably, the longitudinal grooves 10 may be generally parallel to the central longitudinal axis of the tampon. However, the grooves 10 and thereby defined ribs 11 may assume various overall shapes in the axial direction, e.g., they may be straight (linear), sinusoidal, meandering, spiral, helical, hyperbolic, or the like.

Grooves which (or at least a portion of which) extend at an angle with the longitudinal axis of the tampon (e.g., ≧5°, or ≧10° C., preferably ≧20° or ≧30°, more preferably ≧40° or ≧50° C. or ≧60°, even more preferably ≧70°, or ≧80°, or even about 90°) can further improve the contact and grip between the withdrawal end 3 of the tampon and the consumer's fingers. For example, the groove(s) 10 per se may be non-parallel with (i.e., extend at an angle with) the longitudinal axis of the tampon; e.g., the groove(s) 10 may be spiral or slanted. For example, at least a portion of the groove(s) 10 may be non-parallel with (i.e., extend at an angle with) the longitudinal axis of the tampon; e.g., whereas a meandering or sinusoidal groove may overall run in parallel with the longitudinal axis of the tampon, the individual wave-like portions of the groove(s) will be non-parallel with said axis.

A groove 10 may extend over various portions of the circumference of the withdrawal end 3 of the tampon 1. For example, the groove 10 may extend over at least about 5°, 10°, 20°, 30°, 40°, 50°, 60°, 70°, 80°, 90°, 100°, 120° or over more than 120° of the circumference of the withdrawal end 3 of the tampon 1.

The longitudinal grooves 10 may extend along at least 50%, e.g., at least 60%, preferably at least 70%, e.g., at least 80%, more preferably at least 90%, e.g., at least 95% or even about 100% of the axial length of the withdrawal end 3 of the tampon.

The herein described advantageous effects begin to be particularly pronounced when the longitudinal grooves 10 extend along at least 50% of the axial length of the withdrawal end 3 of the tampon. For example, the longitudinal grooves may extend over the relatively more proximal portion of the withdrawal end 3 and/or over the relatively more distal portion of the withdrawal end 3 of the tampon 1. The provision of such grooves at least at or adjacent to the distal edge of the withdrawal end 3 of the tampon 1 can especially facilitate the “opening-out” of said distal edge of the withdrawal end to receive a consumer's fingertip, as well as the grip with consumer's fingers and ejector tube of applicators.

The withdrawal end 3 of the tampon 1 may comprise varying number (even or odd) of such grooves 10. For example, the withdrawal end 3 may comprise between 3 and 12 grooves, e.g., 3, 4, 5, 6, 7, 8, 9, 10, 11 or 12, more preferably between 6 and 12, even more preferably at least about 8 grooves, e.g., 8 grooves. Preferably, where the withdrawal end 3 of the tampon 1 comprises two or more longitudinal grooves, these may be spaced circumferentially at regular intervals, i.e., at regular circumferential angle intervals.

Optionally and preferably, the central portion 4 of the tampon may also comprise one or more radially pressed grooves 12 which delineate adjacent ribs 13. Such ribs 13 are relatively less compressed and extend radially outwardly. The provision of such pressed grooves and ribs on the central portion 4 of the tampon 1 can increase the overall absorption and expansion capacity of tampons.

Preferably, the grooves 12 may be longitudinal, i.e., extending along at least a portion of the axial length of the central section 4 of the tampon 1. Preferably, the longitudinal grooves 12 may be generally parallel to the central longitudinal axis of the tampon. Preferably, the longitudinal grooves 12 may extend along at least 50%, e.g., at least 60%, preferably at least 70%, e.g., at least 80%, more preferably at least 90%, e.g., at least 95% or even about 100% of the axial length of the central portion 4 of the tampon. They may at least partly extend into the insertion end 2 of the tampon.

The grooves 12 and thereby defined ribs 13 may assume various overall shapes in the axial direction, e.g., they may be straight (linear), sinusoidal, meandering, spiral, helical, hyperbolic, or the like. A groove 12 may extend over various portions of the tampon circumference. For example, a groove may extend over at least about 10°, 20°, 30°, 40°, 50°, 60°, 70°, 80°, 90°, 100°, 120°, 130°, 140°, 150° or over more than 150° of the tampon circumference, e.g., over 80° to 120°, or 80° to 150°.

The central portion 4 of the tampon 1 may comprise varying number (even or odd) of such longitudinal grooves 12 and ribs 13, e.g., depending on the diameter of the tampon and/or the type of absorption material, etc. Preferably, the central section 4 of the tampon 1 may comprise between 3 and 12 grooves 12 and/or ribs 13, e.g., 3, 4, 5, 6, 7, 8, 9, 10, 11 or 12, more preferably between 6 and 12, even more preferably at least about 8 grooves or ribs, e.g., 8 grooves and/or ribs. Preferably, where the central portion 4 of the tampon 1 comprises two or more longitudinal grooves, these may be spaced circumferentially at regular intervals, i.e., at regular circumferential angle intervals.

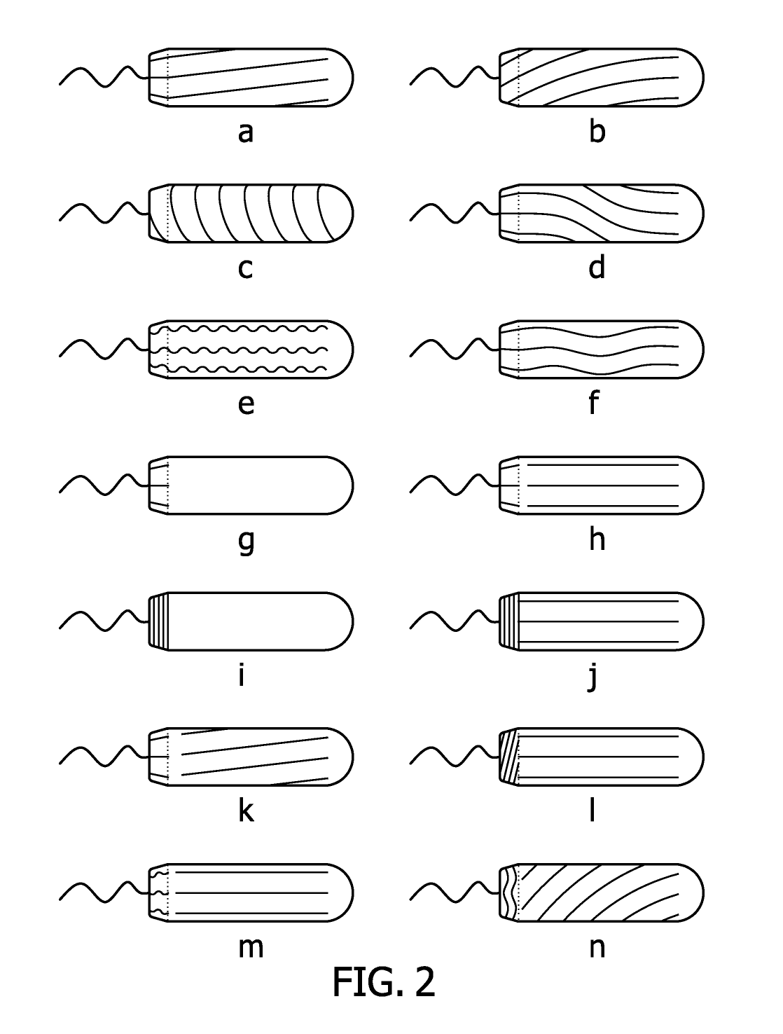

In an embodiment, the grooves 10 of the withdrawal end 3 may be continuous with the grooves 12 of the central portion 4 of the tampon 1. In other words, the grooves 12 of the central portion 4 (partly) extend without interruption into the withdrawal end 3 of the tampon 1. Examples of such configurations are show in FIGS. 1 and 2 a-f, l.

In another embodiment, the grooves 10 of the withdrawal end 3 may be discontinuous with the grooves 12 of the central portion 4 of the tampon 1, or in another embodiment, the central portion may not contain any grooves. Examples of such configurations are show in FIG. 2 g-k, m and n.

In embodiments, the grooves 10 of the withdrawal end 3 and the grooves 12 of the central portion 4 of the tampon 1 may have substantially same or similar shape in the axial direction and/or may extend under substantially same or similar angle relative to the longitudinal axis of the tampon. Examples of such configurations are show in FIGS. 1 and 2 a-f, h.

In embodiments, the grooves 10 of the withdrawal end 3 and the grooves 12 of the central portion 4 of the tampon 1 may have different shapes in the axial direction and/or may extend under different angles relative to the longitudinal axis of the tampon. Examples of such configurations are show in FIG. 2 j-n.

The grooves as taught herein can be brought to the tampon in any manner well known to those of ordinary skill in the art. Typically, tampons comprising grooves 10 within the constricted withdrawal end 3 and optionally also grooves 12 within the central section 4 as explained above can be formed by simultaneous pressing of narrow, strip-shaped sections of the circumferential surface of a tampon blank arranged in a spaced manner to form a number of the desired grooves, preferably longitudinal grooves. The direction of pressing of a groove comprises a radial component of compression, i.e., a component converging toward the central longitudinal axis of the tampon along a radial line, but may also comprise a component of compression diverging from the central longitudinal axis of the tampon (for example, see tampons disclosed in EP 1 547 555, incorporated by reference herein).

Said pressing of the tampon blank produces a highly compressed, solid central part of the tampon, which is typically generally cylindrical with its circumference demarcated by the valley surface of the pressed grooves, and relatively uncompressed ribs interposed between the grooves and extending outwardly from the highly compressed central part.

The depth of the grooves as taught herein may be any, however, it may be preferred that the grooves display a depth of at least about 0.5 mm, preferably of at least about 1 mm, even more preferably of at least about 1.5 mm, such as, e.g., depth between about 2 mm to about 6 mm. Such groove depths particularly ensure the diverse effects as explained herein. The depth of the grooves may vary along the axial length of the grooves or may be substantially invariable along said axial length.

Usually, the outer ends of the ribs may be subsequently pressed to provide a soft, smooth circumferential surface of the tampon, while preserving the relatively uncompressed structure of the ribs, achieving good absorption.

Suitable manners of providing tampons with grooves and ribs of the type intended herein are generally known and are disclosed inter alia in DE 3,934,153 C2, U.S. Pat. No. 5,911,712, WO 02/078586, EP 1 108 408, EP 1 459 720, EP 1 547 555 or U.S. Ser. No. 03/208,180, incorporated by reference herein.

It shall be appreciated that the grooves 10 of the withdrawal end 3 and the grooves 12 of the central section 4 of the tampon 1 may be produced concurrently by the same compressing action, or may be produced by one or more separate compressing actions in any order. The grooves 10 of the withdrawal end 3 may be produced prior to, concurrently with or following the compressing action that forms the constriction of the withdrawal end 3.

Further advantageous features of the tampon as taught herein are contemplated in the following.

In an embodiment, the tampon 1 has a lower expansion capacity in the withdrawal end 3 compared to the remaining tampon body, especially compared to the central section 4 of the tampon. Lower expansion capacity of the withdrawal end may be achieved, e.g., by a higher degree of compression possibly in conjunction with inclusion of a relatively greater amount of absorbent material at the withdrawal end, and/or by increased temperature during compression, etc. Hence, in expanded condition (e.g., after absorbing liquids) the tampon may display a lower diameter in the withdrawal end 3 than in its remaining portions. For example, in an embodiment, the grooves 10 of the withdrawal end 3 may be formed by radial pressure greater than the grooves 12 optionally provided on the remainder of the tampon body, in particular on its central section 4. For example, the pressure used for forming the grooves 10 of the withdrawal end 3 may be between about 1.1 to about 10 times the pressure used for forming the grooves 12 of the central section 4, e.g., between about 2 and 5 times said latter pressure.

In an embodiment, the density and/or hardness of the absorbent material may be smaller in a proximal portion of the withdrawal end 3 of the tampon 1 compared to a more distal portion of said withdrawal end 3. For example, the density and/or hardness of the absorbent material of the withdrawal end 3 of the tampon 1 may decrease, e.g., gradually decrease (such as, e.g., stepwise, linearly, or as a non-linear function), in the proximal direction. Such configuration can provide for a better contact of the relatively less dense and/or softer proximal portion of the withdrawal end 3 with the consumer's fingers, while ensuring less expansion and thereby easier removal of the relatively more dense and/or harder distal portion of the withdrawal end 3. It shall be appreciated that these advantages may also result from said feature even when the constricted withdrawal end does not comprise radially pressed grooves. For example, the density and/or hardness of the absorbent material in a proximal portion of the withdrawal end 3 may be between 0.1 and 0.9 times, e.g., between 0.3 and 0.6 times the density and/or hardness of the absorbent material in a distal portion of the withdrawal end 3.

In another embodiment, the density and/or hardness of the absorbent material may be smaller in a distal portion of the withdrawal end 3 of the tampon 1 compared to a more proximal portion of said withdrawal end 3. For example, the density and/or hardness of the absorbent material of the withdrawal end 3 of the tampon 1 may decrease, e.g., gradually decrease (such as, e.g., stepwise, linearly, or as a non-linear function), in the distal direction. Such configuration can provide for an improved ability of the distal edge of the withdrawal end 3 to open-out and accommodate a user's fingertip, thereby increasing stability of contact there between. It shall be appreciated that these advantages may also result from said feature even when the constricted withdrawal end does not comprise radially pressed grooves. For example, the density and/or hardness of the absorbent material in a distal portion of the withdrawal end 3 may be between 0.1 and 0.9 times, e.g., between 0.3 and 0.6 times the density and/or hardness of the absorbent material in a proximal portion of the withdrawal end 3.

In an embodiment, the angle between the longitudinal axis of the tampon 1 and the side edge of the withdrawal end 3 or tangent thereof may range from 2 to 70°, preferably from 5 to 60°. Such degrees of constrictions of the withdrawal end 3 perform particularly well in the context of the invention.

In an embodiment, the density and/or hardness of the absorbent material may be higher in the constricted withdrawal end 3 compared to the remainder of the tampon body, in particular compared to the central section 4. This can increase the firmness and integrity of the withdrawal end. It shall be appreciated that these advantages may also result from said feature even when the constricted withdrawal end does not comprise radially pressed grooves.

In an embodiment, the density and/or hardness of the absorbent material may be lower in the constricted withdrawal end 3 compared to the remainder of the tampon body, in particular compared to the central section 4. This increases the ability of the withdrawal end to conform and contact a user's fingers. It shall be appreciated that these advantages may also result from said feature even when the constricted withdrawal end does not comprise radially pressed grooves.

The present invention further provides a method for producing a tampon according to the invention. Generally, the method comprises: (a) providing a tampon blank of absorbent material and (b) compressing the tampon blank to provide a tampon as taught herein. For example, the method may comprise: (a) providing a tampon blank of absorbent material and (b) compressing the tampon blank to provide a tampon 1 having a generally elongated body comprising an insertion end 2, a withdrawal end 3 and a central portion 4 extending therebetween, wherein said withdrawal end 3 is constricted, and whereby one or more radially pressed grooves 10 are formed within said constricted withdrawal end 3 of the tampon 1.

A tampon blank as intended herein may be suitably formed by folding or rolling or otherwise manipulating a strip of absorbent material, such as, e.g., a loosely associated and/or tangled fibrous web, into a generally elongated shape, e.g., a generally cylindrical shape.

To form the constricted withdrawal end 3 (e.g., a frusto-conical shape) an optionally the constricted insertion end 2 (e.g., a round-dome shape), the tampon blank in the area of said withdrawal end 3 and optionally said insertion end 2 can be subjected to a higher radial compressing pressure than in the remaining areas of the tampon blank, such as particularly in the central section 4 of the tampon blank.

The method may further comprise providing the absorbent material, the tampon blank or the formed tampon with a withdrawal cord 9. Commonly, the withdrawal cord 9 may be wrapped around a section of absorbent material strip, before it is wound up into the tampon blank.

Particularly when a digital tampon is intended, the method may further comprise providing the withdrawal end 3 of the tampon with a finger recess, e.g., by indenting the finger recess by a compression rod.

In order to provide the withdrawal end 3 and optionally and preferably the central section 4 of the tampon 1 with grooves 10 and 12, respectively, the method comprises compressing the tampon blank on its outer circumferential surface, forming said grooves and preferably a fibre core with a high degree of compression from which relatively uncompressed longitudinal ribs extend radially outward.

To provide the tampon with an outer cover, the method may further comprise applying a strip of a covering and fixing said strip at least to the end of that side of the absorbent material section that lies on the outside during the manufacture of the tampon before the absorbent material section is wound up to form the tampon blank. The covering at least partly covers the circumferential surface of the tampon blank, and preferably does not cover the insertion end.

Describing now an exemplary embodiment of the method in additional detail and without limitation, a preferably cylindrical tampon blank is introduced in a press apparatus. A preferred press apparatus comprises a press having press jaws which are arranged in a star formation with respect to the press axis and can be moved synchronously in a common plane radially with respect to the press axis between their open position and closed position and, in their closed position, are supported on one another on their mutually opposite longitudinal sides. A preferred press consists of eight press jaws which are arranged in a star formation in a common plane at equal angle intervals about and at the same radial distance from the press axis. It is desirable to equip the press with an even number of press jaws, but other numbers of press jaws can be used, including odd numbers. The number of press jaws can vary, for example depending on the weight and the composition of the material intended for the tampon and can also be smaller or greater than eight, although the number generally should not be under four. The press jaws can be moved synchronously radially with respect to the press axis between their open position and closed position.

The press jaws can preferably be heated and preferably each press jaw has its own temperature sensor. By heating the press jaws, it is possible to reduce the memory effect of modern, highly absorbent, greatly expanding fibrous materials, which occurs after the tampon has been finished. By means of the heated press jaws, the surface of the tampon is simultaneously smoothed during pressing and pushing out, and a qualitatively improved surface is produced even in tampons of low weight, the stability of the tampon being preserved. The memory effect of the fibrous material becomes effective again when the fibrous material of the tampon is wetted with body fluid. Each press jaw preferably has a curvature from one end of the press jaw to the other end of the same press jaw. This curvature ensues from the fact that the effective pressing surface of each press jaw has to press a longitudinal groove.

From the above description of the press jaws it is preferred that the press jaws lie diametrically opposite one another in pairs. The present invention also includes the possibility of, in addition to the press jaws described, which produce the fibre core, building press jaws into the press, which serve purposes other than the production of the fibre core. Accordingly, it is possible to use press jaws within the press in order, for example, to stamp patterns or depressions onto or into the surface of the tampon during pressing of the preform, which are intended to serve decorative and/or physical purposes.

The tampon blank is pressed over narrow and preferably identical sections of its circumferential surface at least in the area of the withdrawal end and optionally and preferably also in the area of the central section of the tampon blank. In this way, a preform is produced with grooves, preferably with longitudinal grooves on a solid fibre core with a high degree of compression, which is substantially cylindrical. In the press, the tampon blank is subjected to higher radial compressing pressure in the area of the withdrawal end and optionally and preferably also in the area of the insertion end, than in the remaining areas of the tampon blank, to produce constriction of said end(s).

In an embodiment, to obtain a higher density of fibrous material in the withdrawal end, the tampon blank can additionally either contain more material in the area of the withdrawal end or the material in the withdrawal end should be subject to a higher compression than the insertion end.

In the press, the tampon blank is preferably compressed in a single pressing operation to form the preform, which, on ejection from the press, may be at the same time subjected to final shaping downstream. This final shaping may include a weak radial pressure being exerted on the outer ends of the longitudinal ribs and on the longitudinal grooves. This weak radial pressure has the effect that the outer ends of the mutually opposite side flanks of adjacent longitudinal ribs are pressed against one another so that the longitudinal grooves and thus the outer, approximately cylindrical, soft, closed circumferential surface of the tampon are shaped, and liquid guide ducts are produced in the area of the now radially outwardly closed longitudinal grooves, which ducts are preferably open at least at the insertion end and optionally at the withdrawal end of the tampon. In this way, a considerable increase in the absorption capacity of the tampon is achieved along with very comfortable introduction for the user.

Accordingly, a further aspect of the invention relates to an apparatus, such as a pressing apparatus, specifically designed for manufacturing the tampon as described herein.

It is apparent that there has been provided in accordance with the invention, a tampon product that provides for substantial advantages as set forth above, and other aspects related thereto. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

1. A tampon having a generally elongated body comprising an insertion end, a withdrawal end and a central portion extending therebetween, wherein said withdrawal end is constricted, wherein said constricted withdrawal end comprises one or more radially pressed grooves.

2. The tampon according to claim 1, wherein said one or more grooves define relatively less compressed ribs.

3. The tampon according to claim 1, wherein the one or more grooves or a portion of said one or more grooves extend at an angle with the longitudinal axis of the tampon.

4. The tampon according to claim 1, wherein the one or more grooves are generally longitudinal.

5. The tampon according to claim 1, wherein the central portion of the tampon comprises one or more radially pressed grooves.

6. The tampon according to claim 5, wherein the one or more grooves of the withdrawal end are continuous with the one or more grooves of the central portion of the tampon.

7. The tampon according to claim 5, wherein the one or more grooves of the withdrawal end are discontinuous with the one or more grooves of the central portion of the tampon.

8. The tampon according to claim 1, wherein the density and/or hardness of the absorbent material is smaller in a proximal portion of the withdrawal end of the tampon compared to a distal portion of said withdrawal end.

9. The tampon according to claim 1, wherein the density and/or hardness of the absorbent material is smaller in a distal portion of the withdrawal end of the tampon compared to a proximal portion of said withdrawal end.

10. The tampon according to claim 1, wherein the tampon has a lower expansion capacity in the withdrawal end compared to the remaining tampon body, particularly compared to the central section of the tampon.

11. The tampon according to claim 1, wherein the withdrawal end of the tampon has a frusto-conical shape.

12. The tampon according to claim 1, wherein the tampon comprises a digital tampon.

13. A method for producing the tampon as defined in claim 1, said method comprising: (a) providing a tampon blank of absorbent material and (b) compressing the tampon blank to provide a tampon as defined in claim 1.

14. (canceled)

15. A method of using the tampon as defined in claim 1, said method comprising using the tampon for absorbing a liquid.
